(19) United States
(12) Patent Application Publication
(10) Pub. No.: US 2016/0237005 A1
(43) Pub. Date: Aug. 18, 2016

(54) PROCESS FOR THE OXIDATIVE DEHYDROGENATION OF ETHANE TO ETHYLENE

(71) Applicant: SHELL OIL COMPANY, Houston, TX (US)

(72) Inventors: Dharmesh KUMAR, Bangalore (IN); Ronald Jan SCHOONEBEEK, Amsterdam (NL)

(21) Appl. No.: 15/031,034

(22) PCT Filed: Oct. 24, 2014
(86) PCT No.: PCT/EP2014/072846

§ 371 Date: Apr. 21, 2016

(30) Foreign Application Priority Data
Oct. 25, 2013 (EP) 13190240.5

(51) Int. Cl. C07C 5/48; C07C 29/149; C07C 1/24; C07C 7/04

(52) U.S. Cl. CPC C07C 5/48; C07C 7/04; C07C 29/149; C07C 1/24

(57) ABSTRACT

The present invention provides a process for the oxidative dehydrogenation of ethane to ethylene, the process at least comprising the steps of: (a) providing an ethane-containing stream (10); (b) subjecting the ethane-containing stream (10) provided in step (a) to oxidative dehydrogenation, thereby obtaining a stream (20) containing at least ethylene, water and acetic acid; (c) separating acetic acid from the stream (20) obtained in step (b), thereby obtaining a concentrated acetic acid stream (60) and a first ethylene-enriched stream (40); (d) subjecting the concentrated acetic acid stream (60) obtained in step (c) to hydrogenation thereby obtaining an ethanol-containing stream (80); and (e) subjecting the ethanol-containing stream (80) obtained in step (d) to dehydration thereby obtaining a second ethylene-enriched stream (90).

The present invention relates to a process for the oxidative dehydrogenation of ethane to ethylene. Processes for the oxidative dehydrogenation (ODH) of ethane to ethylene are known in the art and are seen as a potential alternative to current ethylene production processes such as steam cracking and autothermal cracking (ATC) processes. Steam cracking and ATC processes produce a variety of other products than ethylene (hence requiring subsequent separation) and require a high energy input.

In processes for the oxidative dehydrogenation of ethane, ethylene is the main product with typically small amounts of carbon monoxide (CO) and carbon dioxide (CO₂) as byproducts. An example of a known ODH process is disclosed in US 2010/0256432, the teaching of which is hereby incorporated by reference.

A problem of known processes for the oxidative dehydrogenation of ethane is that significant amounts of acetic acid are formed, resulting in reduced ethylene selectivity. Usually, the acetic acid (if the acetic acid is not one of the intended products and if ethylene selectivity is to be imporved) needs to be neutralized using for example sodium hydroxide before hydrogenation takes place.

It is an object of the present invention to overcome or minimize the above problem.

It is a further object of the present invention to provide an alternative process for the oxidative dehydrogenation of ethane to ethylene.

One or more of the above or other objects can be achieved by providing a process for the oxidative dehydrogenation of ethane to ethylene, the process at least comprising the steps of:

(a) providing an ethane-containing stream;

(b) subjecting the ethane-containing stream provided in step (a) to oxidative dehydrogenation, thereby obtaining a stream containing at least ethylene, water and acetic acid;

(c) separating acetic acid from the stream obtained in step (b), thereby obtaining a concentrated acetic acid stream and a first ethylene-enriched stream;

(d) subjecting the concentrated acetic acid stream obtained in step (c) to hydrogenation thereby obtaining an ethanol-containing stream; and

(e) subjecting the ethanol-containing stream obtained in step (d) to dehydration thereby obtaining a second ethylene-enriched stream.

It has surprisingly been found that the process according to the present invention results in increased ethylene production, as any acetic acid formed during the process may be converted to ethylene.

In step (a), an ethane-containing stream is provided. Preferably, the ethane-containing stream provided in step (a) comprises from 5 to 95 vol. % ethane, preferably at least 50 vol. %, more preferably at least 60 vol. %. The amount of ethane in the ethane-containing stream will typically depend on e.g. the origin of the ethane-containing stream and on whether air or a more pure O₂-stream is used in the oxidative dehydrogenation step in step (b).

Although the person skilled in the art will readily understand that O₂ can be added separately as well, it is preferred that O₂ (either in the form of air or a more concentrated O₂-stream) has been added to the ethane-containing stream provided in step (a), before subjecting the stream to the oxidative dehydrogenation step in step (b). In the latter case, the ethane-containing stream provided in step (a) comprises from 1 to 40 vol. % O₂, preferably at most 30 vol. %, more preferably at most 25 vol. %. Further it is preferred that the ethane-containing stream provided in step (a) comprises at most 10 vol. % N₂, preferably at most 5.0 vol. %, more preferably at most 1.0 vol. %. Most preferably, the ethane-containing stream provided in step (a) comprises no N₂ at all.

In step (b), the ethane-containing stream provided in step (a) is subjected to oxidative dehydrogenation (ODH), thereby obtaining a stream containing at least ethylene, water and acetic acid. As the person skilled in the art is familiar with the oxidative dehydrogenation of ethane (including selecting appropriate catalyst(s) and conditions), this is not discussed here in full detail. As a mere example, the oxidative dehydrogenation of ethane has been described in M. M. Bhasin et al., “Dehydrogenation and oxydehydrogenation of paraffins to olefins”, Applied Catalysis A: General 221 (2001), 397-419, the teaching of which is hereby incorporated by reference. Typically, the temperature during oxidative dehydrogenation in step (b) is between 300 and 450° C. and the pressure is typically between 0.1 and 40 bara.

Preferably, the stream obtained in step (b) comprises an amount of acetic acid which is from 0.5 to 10% of the amount of ethane as present in the ethane-containing stream provided in step (a), preferably at most 5.0%.

In step (c), acetic acid is separated from the stream obtained in step (b), thereby obtaining a concentrated acetic acid stream and a first ethylene-enriched stream. Typically, the separation of the acetic acid from the stream obtained in step (d) is performed by separation (for example by condensation of water and acetic acid) of the water/acetic acid and subsequent distillation thereof. As the person skilled in the art readily understands how to achieve this, this is not further discussed in detail.

Preferably, the concentrated acetic acid stream obtained in step (c) comprises at least 5 vol. % acetic acid, preferably at least 10 vol. %, more preferably at least 50 vol. %, or even at least 80 vol. % or at least 95 vol. %.

In step (d), the concentrated acetic acid stream obtained in step (c) is subjected to hydrogenation thereby obtaining an ethanol-containing stream. As the person skilled in the art readily understands how to achieve this, this is not further discussed in detail.

Preferably, in the hydrogenation of step (d) hydrogen (H₂) is used that has been produced in a steam cracker.

In step (e), the ethanol-containing stream obtained in step (d) is subjected to dehydration thereby obtaining a second ethylene-enriched stream.

As the person skilled in the art readily understands how to achieve this, this is not further discussed in detail.

According to a preferred embodiment of the process according to the present invention, the first ethylene-enriched stream obtained in step (c) and the second ethylene-enriched stream obtained in step (e) are combined thereby obtaining a combined ethylene-enriched stream.

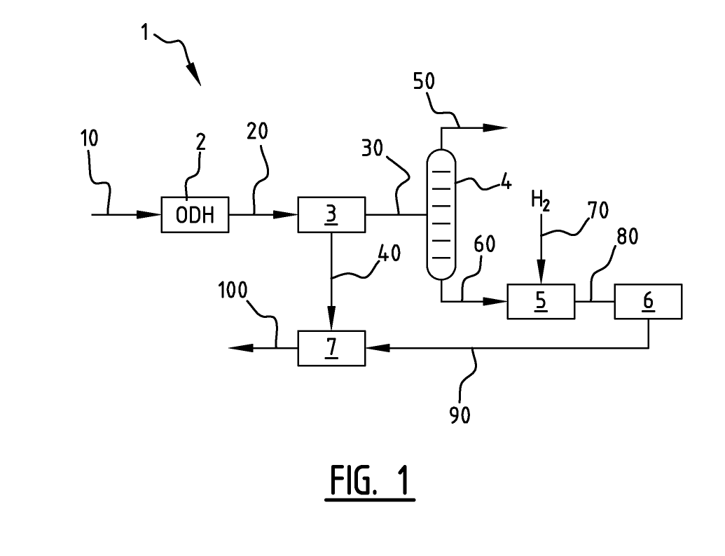

Hereinafter the invention will be further illustrated by the following non-limiting drawing. FIG. 1 schematically shows a process scheme for the oxidative dehydrogenation of ethane to ethylene. The process scheme is generally referred to with reference number 1.

The process scheme 1 comprises an oxidative dehydrogenation reactor 2, a separator 3, a distillation column 4, a hydrogenation reactor 5, a dehydration reactor 6 and a vessel 7.

During use, an ethane-containing stream 10 is fed to the oxidative dehydrogenation reactor 2. In the embodiment of FIG. 1, oxygen (e.g. in the form of air or a more concentrated O₂-stream) has been added to the ethane-containing stream 10 before feeding it to the oxidative dehydrogenation reactor 2. In the oxidative dehydrogenation reactor 2 a stream 20 containing at least ethylene, water and acetic acid is obtained. Stream 20 is fed to separator 3 to obtain a first ethylene-enriched stream 40 and a stream 30 rich in water and acetic acid. Stream 30 is separated in distillation column 4 thereby obtaining a water-enriched stream 50 and a concentrated acetic acid stream 60. The concentrated acetic acid stream 60 is subjected to hydrogenation in hydrogenation reactor 5 thereby obtaining an ethanol-containing stream 80. The ethanol-containing stream 80 is subjected to dehydration in dehydration reactor 6 thereby obtaining a second ethylene-enriched stream 90.

Preferably, the hydrogen stream 70 as used in the hydrogenation reactor 5 of step (d) has been produced in a nearby steam cracker (not shown).

As shown in the embodiment of FIG. 1, the first ethylene-enriched stream 40 obtained in the separator 3 and the second ethylene-enriched stream 90 obtained in the dehydration reactor 6 are combined (in vessel 7). The combined ethylene-enriched stream 100 may be further treated (e.g. to remove certain trace components).

The person skilled in the art will readily understand that many modifications may be made without departing from the scope of the invention.

1. A process for the oxidative dehydrogenation of ethane to ethylene, the process at least comprising the steps of:
(a) providing an ethane-containing stream;
(b) subjecting the ethane-containing stream provided in step (a) to oxidative dehydrogenation, thereby obtaining a stream containing at least ethylene, water and acetic acid;
(c) separating acetic acid from the stream obtained in step (b), thereby obtaining a concentrated acetic acid stream and a first ethylene-enriched stream;
(d) subjecting the concentrated acetic acid stream obtained in step (c) to hydrogenation thereby obtaining an ethanol-containing stream; and
(e) subjecting the ethanol-containing stream obtained in step (d) to dehydration thereby obtaining a second ethylene-enriched stream.

2. The process according to claim 1, wherein the ethane-containing stream provided in step (a) comprises from 5 to 95 vol. % ethane.

3. The process according to claim 1, wherein the ethane-containing stream 404 provided in step (a) comprises from 1 to 40 vol. % O₂.

4. The process according to claim 1, wherein the ethane-containing stream provided in step (a) comprises at most 10 vol. % N₂.

5. The process according to claim 1, wherein the stream obtained in step (b) comprises an amount of acetic acid which is from 0.5 to 10% of the amount of ethane as present in the ethane-containing stream provided in step (a).

6. The process according to claim 1, wherein the concentrated acetic acid stream obtained in step (c) comprises at least 5 vol. % acetic acid.

7. The process according to claim 1, wherein in the hydrogenation of step (d) hydrogen (H₂) is used that has been produced in a steam cracker.

8. The process according to claim 1, wherein the first ethylene-enriched stream obtained in step (c) and the second ethylene-enriched stream obtained in step (e) are combined thereby obtaining a combined ethylene-enriched stream.
